(19) United States
(12) Patent Application Publication
(10) Pub. No.: US 2023/0260633 A1
(43) Pub. Date: Aug. 17, 2023

(54) ADVANCED PATIENT REGISTRATION AND AUDIT SYSTEM

(71) Applicant: MPN Software Systems, Inc., Upper Saddle River, NJ (US)

(72) Inventor: Michael Norworth, Upper Saddle River, NJ (US)

(73) Assignee: MPN Software Systems, Inc., Upper Saddle River, NJ (US)

(21) Appl. No.: 18/109,685

(22) Filed: Feb. 14, 2023

Related U.S. Application Data
(60) Provisional application No. 63/310,727, filed on Feb. 16, 2022.

(51) Int. Cl. G16H 40/20; G16H 10/20; G16H 10/60; G06K 7/14

(52) U.S. Cl. CPC G16H 40/20; G16H 10/20; G16H 10/60; G06K 7/1417

(57) ABSTRACT

Systems, methods, and instrumentalities are disclosed herein for allowing a patient to check in for a visit to a provider while creating a record of the patient being physically present at the provider's office at the time of the visit. The patient may scan a QR code associated with the provider using a mobile device. The patient's mobile device may download a questionnaire that may include one or more facility-specific questions, and may prompt the patient to enter biographical information and information. The mobile device may transmit the patient's responses and a unique ID associated with the mobile device, to a cloud server. The cloud server may transmit the responses and the unique ID to a local server in the provider's office. The local server may add the responses and/or the unique ID to a patient record associated with the patient and/or may log the information in an audit log.

### CROSS-REFERENCE TO RELATED APPLICATIONS

This application claims the benefit of U.S. Provisional Patent Application No. 63/310,727, filed Feb. 16, 2022, the contents of which are incorporated herein by reference in their entirety.

### BACKGROUND

Different methods may be used to check in a patient prior to a provider visit. For example, the provider (or the provider's assistant) may interview the patient face-to-face, recording the patient's information on paper or via a mobile device. Alternatively, the patient may use a mobile device that is provided by the provider's office to check in. However, these methods may expose the patient to germs, either from the provider themself or from other patients who have used the mobile device to check in previously. Due to the prevalence of the coronavirus disease 2019 (COVID-19), among others, patients may be hesitant to check in using these methods.

Conducting a face-to-face interview or using the provider's mobile device may create a record of the patient being physically present in the provider's office at a given time (for example by using a manual sign-in sheet), but this might expose the patient to the germs of others. Simple software solutions like using a magnetic card-swipe system may be inadequate to provide a verifiable, independent record of a patient being physically present in the provider's office. Thus, a solution that both allows the patient to avoid exposure to harmful diseases and creates an independently verifiable record of the patient being physically present at the provider's office is needed.

### SUMMARY

Systems, methods, and instrumentalities are disclosed herein for allowing a patient to check in for a visit to a provider while creating a record of the patient being physically present at the provider's office at the time of the visit. For example, the provider may use a software package that is executed on one or more computing devices to implement one or more of the systems, methods, and/or instrumentalities disclosed herein.

For example, the provider may use the software to generate a QR code associated with the provider (e.g., the physical location of the provider's office). The QR code may be generated via the software and may be printed onto paper, such that the QR code is physically displayed in the provider's office. The patient may scan the QR code using an application running on the patient's mobile device. In another example, a mobile device provided by the provider may be used (e.g., instead of the patient's mobile device), for example if the patient does not have a compatible mobile device. After the QR code is scanned, the patient's mobile device may download a questionnaire associated with the provider. For example, the questionnaire may include one or more facility-specific questions, and may prompt the patient to enter biographical information and information related to the patient's condition. The patient may fill out the questionnaire, and may respond to the facility-specific questions.

Once the patient has completed the questionnaire, the patient's mobile device may transmit the patient's responses, as well as a unique ID associated with the mobile device, to a cloud server. For example, the unique ID may be an electronic serial number (ESN), an international mobile equipment identity (IMEI), or a mobile equipment identifier (MEID). The cloud server may then transmit the responses and the unique ID to a local server located in the provider's office. Once the responses and the unique ID have been uploaded to the local server, they may be deleted from the cloud server. The local server may add the responses and/or the unique ID to a patient record associated with the patient.

### BRIEF DESCRIPTION OF THE DRAWINGS

FIG. 1A is a diagram of an example load control system.

FIG. 1B is a block diagram illustrating an example of a device capable of processing and/or communication in the load control system of FIG. 1A.

FIG. 1C is a block diagram illustrating an example mobile device.

FIG. 2 is a flowchart of an example procedure for checking in a patient to a healthcare facility.

FIG. 3 is a flowchart of another example procedure for checking in a patient to a healthcare facility.

FIG. 4 is a flowchart of another example procedure for checking in a patient to a healthcare facility.

FIG. 5 is a flowchart of another example procedure for checking in a patient to a healthcare facility.

FIG. 6A is an example QR code that may be used as part of a procedure for checking in a patient to a healthcare facility.

FIGS. 6B-6O are example graphical user interfaces that may be displayed by a mobile device as part of a procedure for checking in a patient to a healthcare facility.

### DETAILED DESCRIPTION

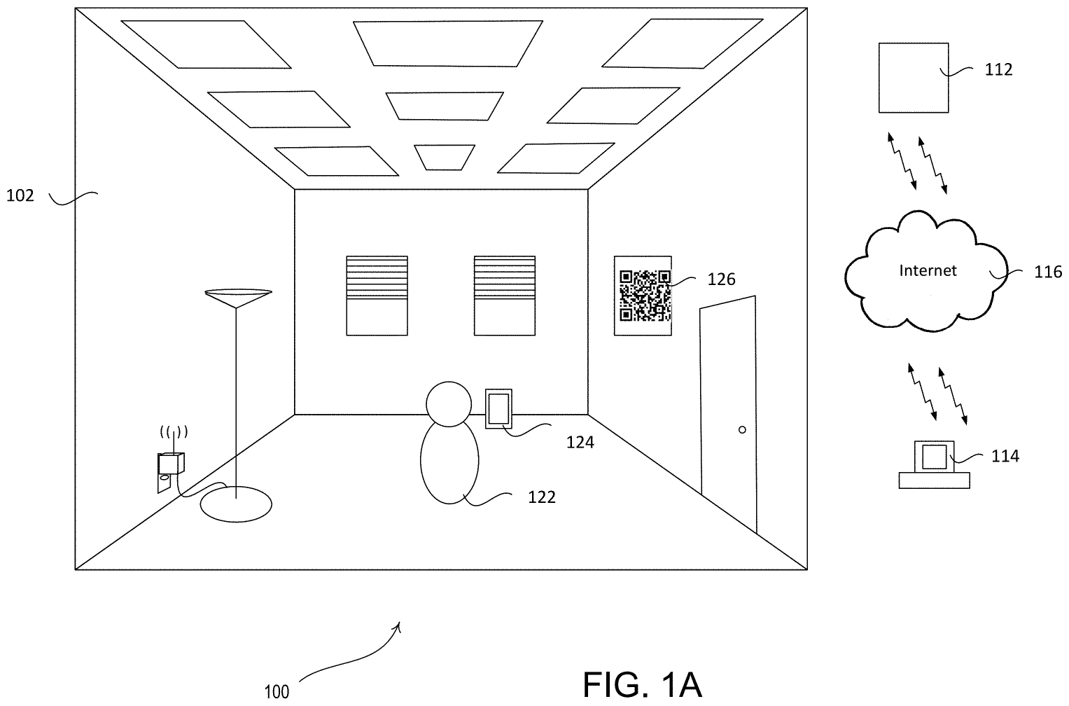

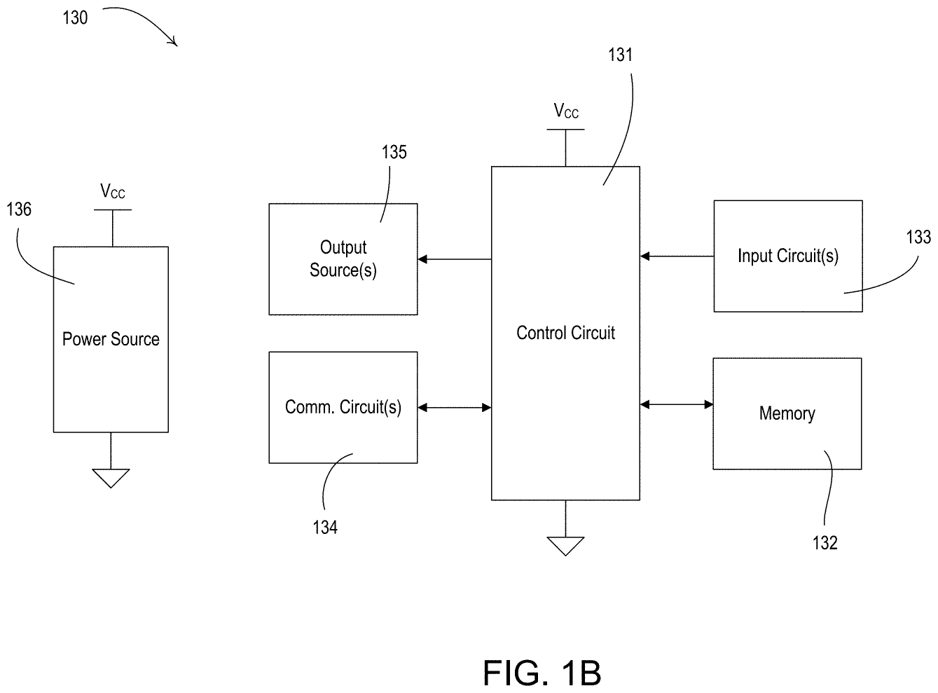

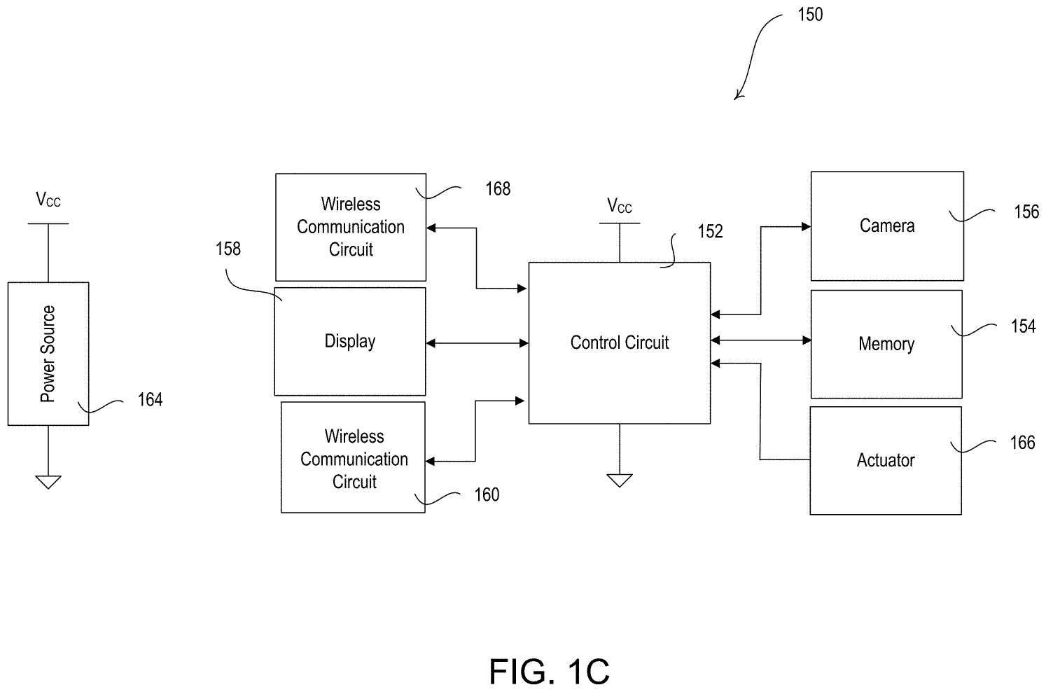

FIG. 1A is a diagram of an example load control system 100. For example, some or all of the load control system 100 may be located within a room 102, which may be part of a healthcare facility. The load control system 100 may include a cloud server (e.g., a remote server) 112, a local server 114, and/or a mobile device 124 associated with a user 122. In another example, the mobile device 124 may be associated with the healthcare facility. The cloud server 112, the local server 114, and/or the mobile device 124 may communicate with each other via the internet 116 (e.g., using a WI-FI® network, a cellular network, a WI-MAX® network, etc.).

The load control environment may include a QR code 126. The user 122 may scan the QR code 126 using a camera of the mobile device 124. The mobile device 124 may transmit a request for data associated with the QR code 126 to the cloud server 112 (e.g., via the internet 116). The cloud server 112 may receive the request and may transmit the data associated with the QR code 126 to the mobile device 124. For example, the data associated with the QR code 126 may include information about a clinic associated with the load control system 100 and/or a plurality of questions, which may include one or more facility-specific (e.g., unique) questions. The provider(s) and/or the staff of the healthcare facility may generate the facility specific questions and may save them to the local server 114. The local server 114 may then transmit the facility-specific questions to the cloud server 112 via the internet 116. The cloud server 112 may then transmit the facility-specific questions to the mobile device 124 when the cloud server 112 receives the request from the mobile device 124.

The mobile device 124 may receive the data associated with the QR code 126, and may prompt the user 122 to enter responses to the plurality of questions. Once the user 122 has entered responses, the mobile device 124 may transmit the responses, along with a unique ID of the mobile device 124 and/or a timestamp at which the responses were transmitted, to the cloud server 114 via the internet 116. The cloud server 112 may then transmit the information received from the mobile device 124 (e.g., and/or a timestamp at which the information was received from the mobile device 124) to the local server 112 via the internet 116. The local server 114 (e.g., and/or the cloud server 112) may determine a patient record that matches with information provided by the user 122. For example, the responses to the plurality of questions may include an email address associated with the user 122, the user 122's date of birth (DOB), and/or a phone number associated with the user 122. The local server 114 may also determine that the user 122 and/or the patient record is associated with an upcoming appointment. The local server 114 may mark the user 122 as having checked in for the upcoming appointment, and/or may store the unique ID of the mobile device 124 and a timestamp in an audit log.

FIG. 1B is a block diagram illustrating an example of a device capable of processing and/or communication in the load control system of FIG. 1A. In an example, the device 130 may be a control device capable of transmitting or receiving messages. The device 130 may be a computing device, such as the mobile device 124, the cloud server 112, the local server 114, a processing device, a central computing device, and/or another computing device in the load control system 100.

The device 130 may include a control circuit 131 for controlling the functionality of the device 130. The control circuit 131 may include one or more general purpose processors, special purpose processors, conventional processors, digital signal processors (DSPs), microprocessors, integrated circuits, a programmable logic device (PLD), application specific integrated circuits (ASICs), and/or the like. The control circuit 131 may perform signal coding, data processing, image processing, power control, input/output processing, and/or any other functionality that enables the device 131 to perform as one of the devices of the load control system (e.g., load control system 100) described herein.

The control circuit 131 may be communicatively coupled to a memory 132, and may store information in and/or retrieve information from the memory 132. The memory 132 may comprise computer-readable storage media and/or machine-readable storage media that maintains a device dataset of associated device identifiers, network information, and/or computer-executable instructions for performing as described herein. For example, the memory 132 may comprise computer-executable instructions or machine-readable instructions that include one or more portions of the procedures described herein. The control circuit 131 may access the instructions from memory 132 for being executed to cause the control circuit 131 to operate as described herein, or to operate one or more other devices as described herein. The memory 132 may comprise computer-executable instructions for executing configuration software and/or control software. The computer-executable instructions may be executed to perform one or more procedures described herein.

The memory 132 may include a non-removable memory and/or a removable memory. The non-removable memory may include random-access memory (RAM), read-only memory (ROM), a hard disk, and/or any other type of non-removable memory storage. The removable memory may include a subscriber identity module (SIM) card, a memory stick, a memory card, and/or any other type of removable memory. The memory 132 may be implemented as an external integrated circuit (IC) or as an internal circuit of the control circuit 131.

The device 130 may include one or more communication circuits 134 that are in communication with the control circuit 131 for sending and/or receiving information as described herein. The communication circuit 134 may perform wireless and/or wired communications. The communication circuit 134 may be a wired communication circuit capable of communicating on a wired communication link. The wired communication link may include an Ethernet communication link, an RS-485 serial communication link, a 0-10 volt analog link, a pulse-width modulated (PWM) control link, a Digital Addressable Lighting Interface (DALI) digital communication link, and/or another wired communication link. The communication circuit 134 may be configured to communicate via power lines (e.g., the power lines from which the device 130 receives power) using a power line carrier (PLC) communication technique. The communication circuit 134 may be a wireless communication circuit including one or more RF or infrared (IR) transmitters, receivers, transceivers, and/or other communication circuits capable of performing wireless communications.

Though a single communication circuit 134 is illustrated in FIG. 1B, multiple communication circuits may be implemented in the device 130. The device 130 may include a communication circuit configured to communicate via one or more wired and/or wireless communication networks and/or protocols, and at least one other communication circuit configured to communicate via one or more other wired and/or wireless communication networks and/or protocols. For example, a first communication circuit may be configured to communicate via a wired or wireless communication link, while another communication circuit may be capable of communicating on another wired or wireless communication link. The first communication circuit may be configured to communicate via a first wireless communication link (e.g., a wireless network communication link) using a first wireless protocol (e.g., a wireless network communication protocol), and the second communication circuit may be configured to communicate via a second wireless communication link (e.g., a short-range or direct wireless communication link) using a second wireless protocol (e.g., a short-range wireless communication protocol).

One of the communication circuits 134 may comprise a beacon transmitting and/or receiving circuit capable of transmitting and/or receiving beacon messages via a short-range RF signal. The control circuit 131 may communicate with beacon transmitting circuit (e.g., a short-range communication circuit) to transmit beacon messages. The beacon transmitting circuit may communicate beacons via RF communication signals, for example. The beacon transmitting circuit may be a one-way communication circuit (e.g., the beacon transmitting circuit is configured to transmit beacon messages) or a two-way communication circuit capable of receiving information on the same network and/or protocol on which the beacons are transmitted (e.g., the beacon transmitting circuit is configured to transmit and receive beacon messages). The information received at the beacon transmitting circuit may be provided to the control circuit 131.

The control circuit 131 may be in communication with one or more input circuits 133 from which inputs may be received. The input circuits 133 may be included in a user interface for receiving inputs from the user. For example, the input circuits 133 may include an actuator (e.g., a momentary switch that may be actuated by one or more physical buttons) that may be actuated by a user to communicate user input or selections to the control circuit 131. The actuator may include a touch sensitive surface, such as a capacitive touch surface, a resistive touch surface, an inductive touch surface, a surface acoustic wave (SAW) touch surface, an infrared touch surface, an acoustic pulse touch surface, and/or another touch sensitive surface that is configured to receive inputs (e.g., touch actuations/inputs), such as point actuations or gestures from a user. The control circuit 131 of the device 130 may enter a mode, transmit a message, transmit control instructions, and/or perform other functionality in response to an actuation or input from the user on the touch sensitive surface.

The control circuit 131 may be in communication with one or more output sources 135. The output sources 135 may include one or more indicators (e.g., visible indicators, such as LEDs) for providing indications (e.g., feedback) to a user. The output sources 135 may include a display (e.g., a visible display) for providing information (e.g., feedback) to a user. The control circuit 131 and/or the display may generate a graphical user interface (GUI) generated via software for being displayed on the device 130 (e.g., on the display of the device 130).

The user interface of the device 130 may combine features of the input circuits 133 and the output sources 135. For example, the user interface may have buttons that actuate the actuators of the input circuits 133 and may have indicators (e.g., visible indicators) that may be illuminated by the light sources of the output sources 135. In another example, the display and the control circuit 131 may be in two-way communication, as the display may display information to the user and include a touch screen capable of receiving information from a user. The information received via the touch screen may be capable of providing the indicated information received from the touch screen as information to the control circuit 131 for performing functions or control.

Each of the hardware circuits within the device 130 may be powered by a power source 136. The power source 136 may include a power supply configured to receive power from an alternating-current (AC) power supply or direct-current (DC) power supply, for example. In addition, the power source 136 may comprise one or more batteries. The power source 136 may produce a supply voltage V_{CC} for powering the hardware within the device 130.

FIG. 1C is a block diagram illustrating an example mobile device 150 (e.g., the mobile device 124 shown in FIG. 1A) as described herein. Though the mobile device 150 is described herein separately from the computing device 130, the mobile device 150 may be a computing device. The block diagram of FIG. 1C may show additional portions of the mobile device 150 that may be implemented in the mobile device and/or other computing devices herein. The mobile device 150 may include a control circuit 152 for controlling the functionality of the mobile device 150. The control circuit 152 may include one or more general purpose processors, special purpose processors, conventional processors, digital signal processors (DSPs), microprocessors, integrated circuits, a programmable logic device (PLD), application specific integrated circuits (ASICs), and/or the like. The control circuit 152 may perform signal coding, data processing, power control, image processing, input/output processing, and/or any other functionality that enables the mobile device 150 to perform as described herein.

The control circuit 152 may store information in and/or retrieve information from the memory 154. The memory 154 may include a non-removable memory and/or a removable memory. The non-removable memory may include random-access memory (RAM), read-only memory (ROM), a hard disk, and/or any other type of non-removable memory storage. The removable memory may include a subscriber identity module (SIM) card, a memory stick, a memory card (e.g., a digital camera memory card), and/or any other type of removable memory.

The mobile device 150 may include a camera 156 that may be in communication with the control circuit 152. The camera 156 may include a digital camera or other optical device configured to generating images or videos (e.g., image sequences) for being captured at the mobile device 150 using visible light. The camera 156 may include a light configured to flash, modulate, and/or turn on/off in response to signals received from the control circuit.

The mobile device 150 may include a first wireless communication circuit 160 for transmitting and/or receiving information. The first wireless communication circuit 160 may perform wireless communications on a first wireless communication link and/or network (e.g., a network wireless communication link). The mobile device 150 may also, or alternatively, include a second wireless communication circuit 168 for transmitting and/or receiving information. The second wireless communication circuit 168 may perform wireless communications via a second wireless communication link and/or network (e.g., a short-range wireless communication link). The first and second wireless communication circuits 160, 168 may be in communication with control circuit 152. The wireless communication circuits 160 and 168 may include RF transceivers and/or other communications modules configured to performing wireless communications via an antenna. The wireless communication circuit 160 and/or the wireless communication circuit 168 may be configured to perform communications via the same communication channels or different communication channels. For example, the first wireless communication circuit 160 may be configured to communicate (e.g., with control devices and/or other devices in the load control system) via the first wireless communication link and/or network using a first wireless communication protocol (e.g., a network wireless communication protocol, such as the CLEAR CONNECT and/or THREAD protocols), and the second wireless communication circuit 168 may be configured to communicate (e.g., with a sensor device or another device) via the second wireless communication channel and/or network using a second wireless communication protocol (e.g., a short-range wireless communication protocol, such as the BLUETOOTH and/or BLUETOOTH LOW ENERGY (BLE) protocols).

The control circuit 152 may also be in communication with a display 158. The display 158 may provide information to a user in the form of a graphical and/or textual display. The control circuit 152 may signal the display 158, or portions thereof, to modulate or turn on/off to communicate information from the display 158. The communication between the display 158 and the control circuit 152 may be a two-way communication, as the display 158 may include a touch screen module configured to receiving information from a user and providing such information to the control circuit 152.

The mobile device 150 may include an actuator 166. The control circuit 152 may be responsive to the actuator 166 for receiving a user input. For example, the control circuit 152 may be operable to receive a button press from a user on the mobile device 150 for making a selection or performing other functionality on the mobile device 150.

One or more of the circuits within the mobile device 150 may be powered by a power source 164. The power source 164 may include an AC power supply or DC power supply, for example. The power source 164 may generate a DC supply voltage V_{CC} for powering the circuits within the mobile device 150.

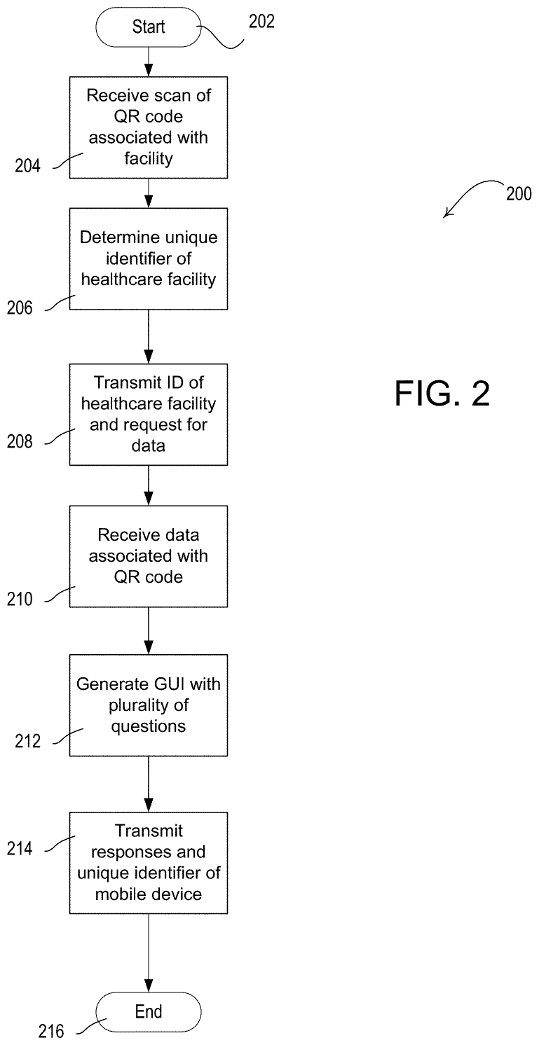

FIG. 2 is a flowchart of an example procedure 200 for checking in a patient to a healthcare facility. The procedure 200 may be performed by a mobile device, such as the mobile device 124 shown in FIG. 1A, and/or another device in the load control system. The mobile device may be associated with a user, such as the user 122 shown in FIG. 1A. Though the procedure 200 may be described herein as being performed by a single device, such as a mobile device, the procedure 200 may be distributed across multiple devices.

The procedure 200 may begin at 202. At 204, the mobile device may receive a scan of a QR code associated with a facility (e.g., a healthcare facility with one or more providers). The mobile device may receive the scan of the QR code via a camera of the mobile device. For example, the user may point the camera of the mobile device at the QR code, and the mobile device may scan the QR code. The QR code may be printed or otherwise accessible in the facility. At 206, the mobile device may determine the unique identifier (unique ID) of the healthcare facility based on the QR code. At 208, the mobile device may transmit the unique identifier of the healthcare facility and a request for data associated with the QR code to a first server (e.g., a cloud server). At 210, the mobile device may receive the data associated with the QR code from the cloud server. The data associated with the QR code may include a plurality of questions to be answered by the user of the mobile device. The plurality of questions may include, for example, one or more facility-specific questions. The mobile device may receive data that identifies the healthcare facility from the cloud server.

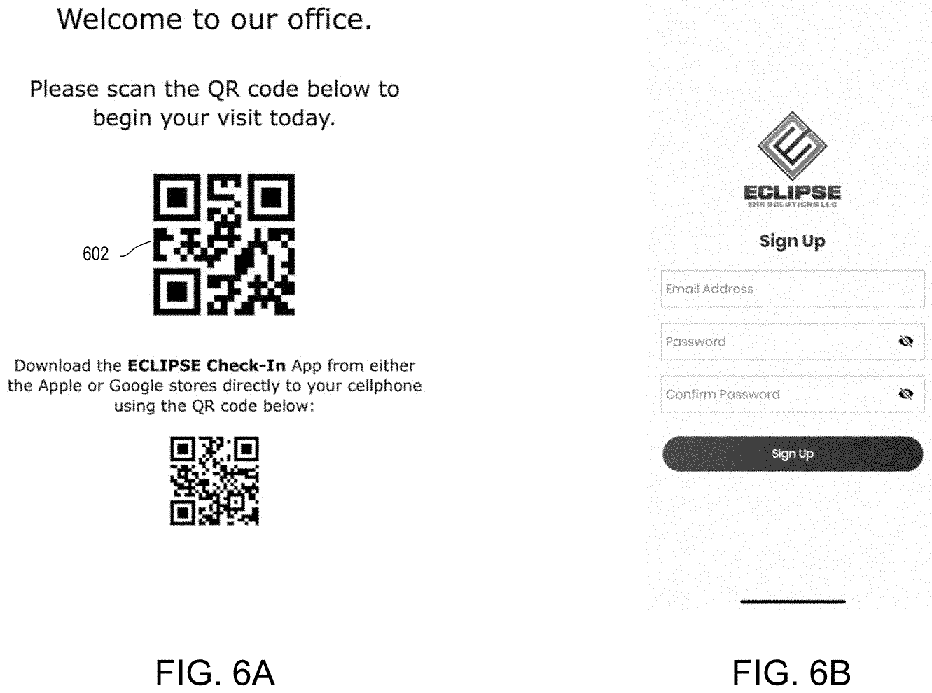

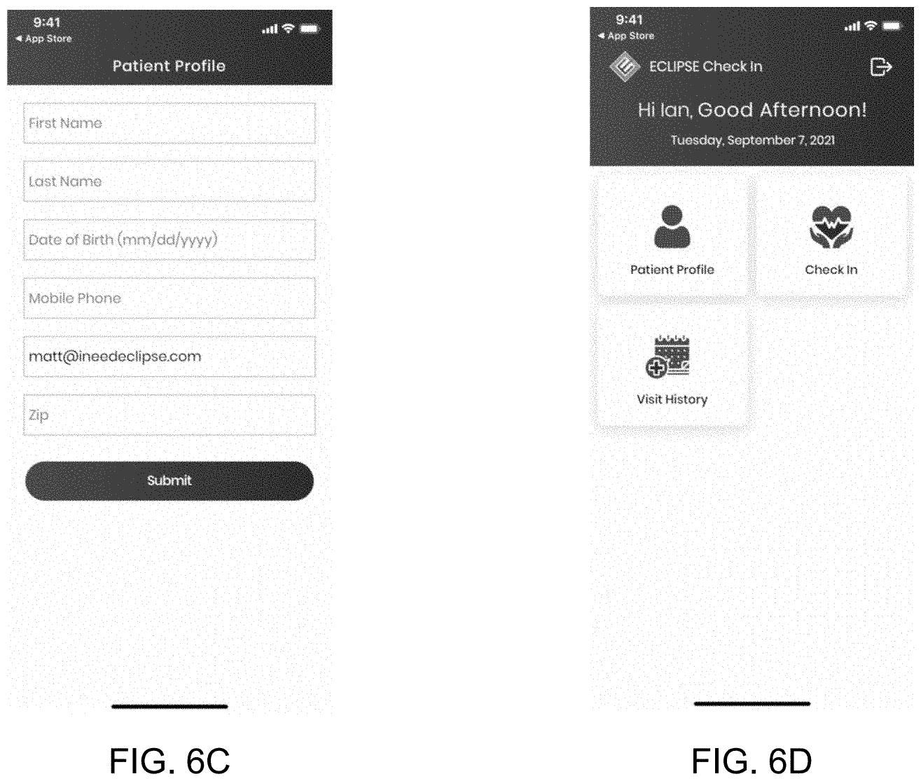

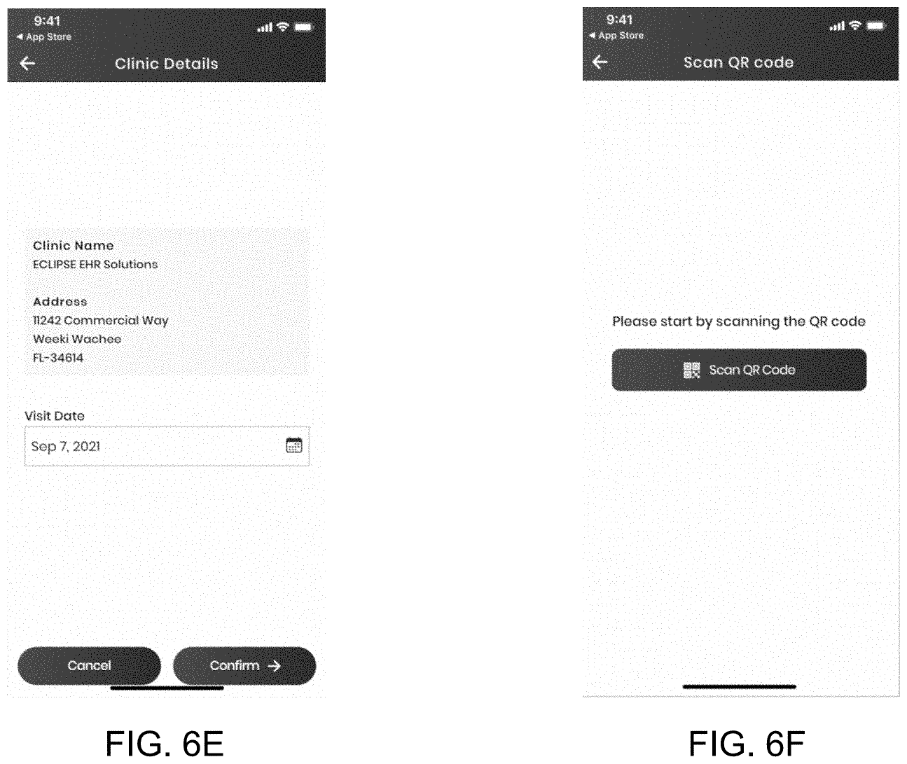

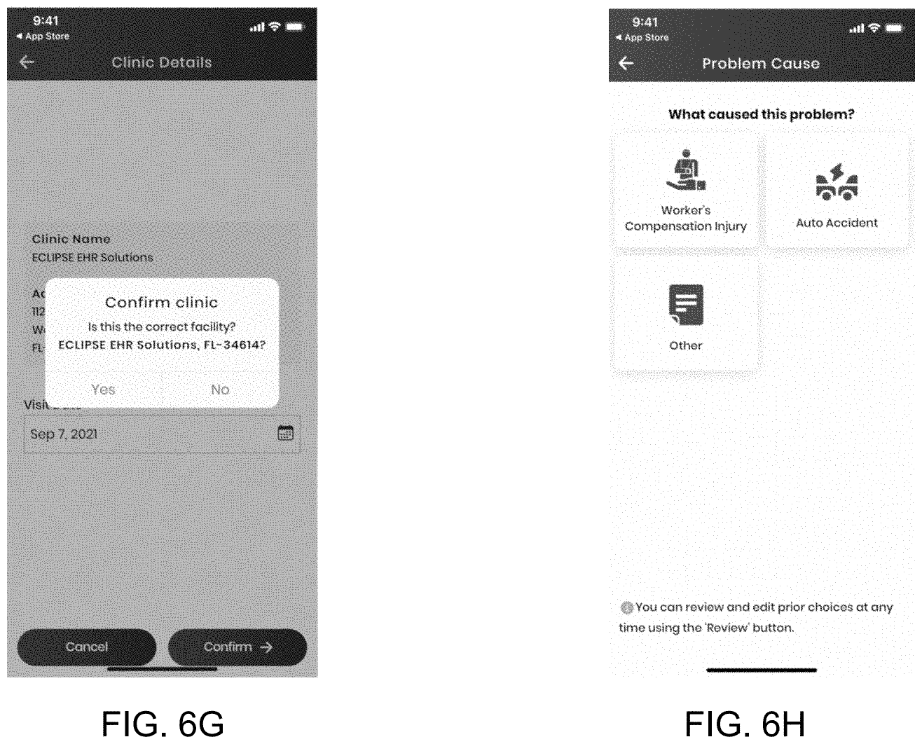

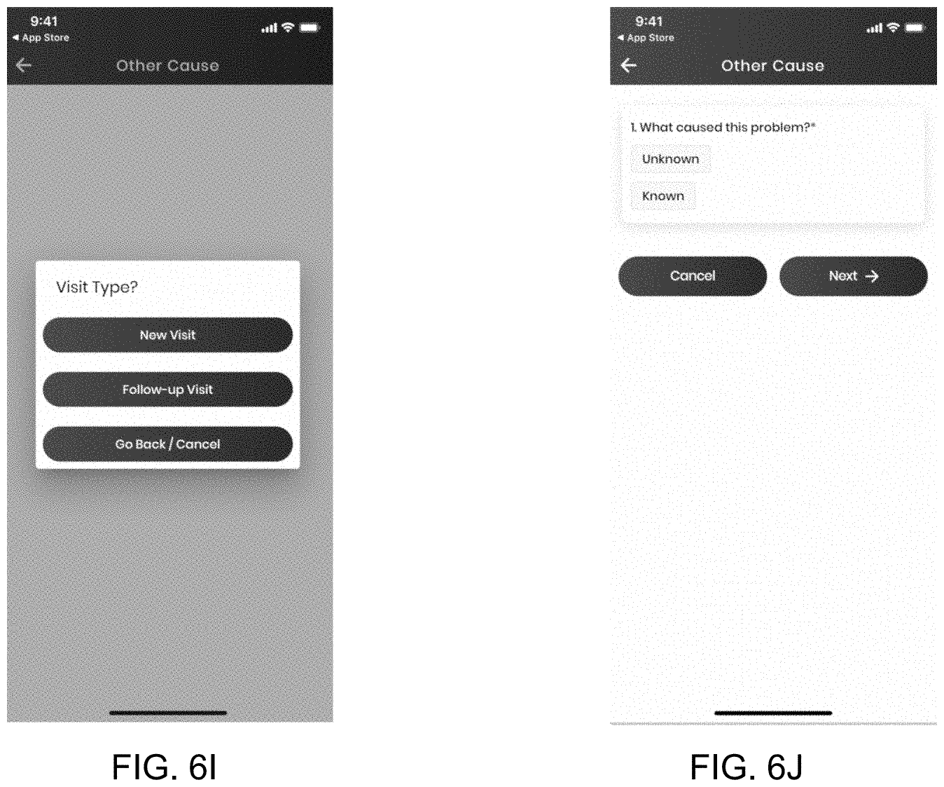

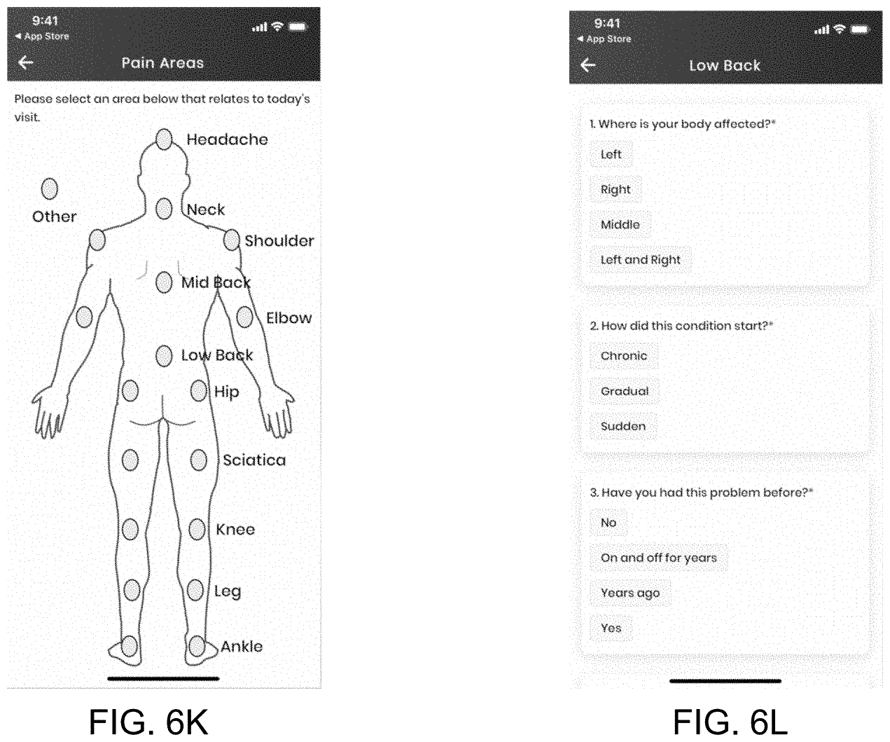

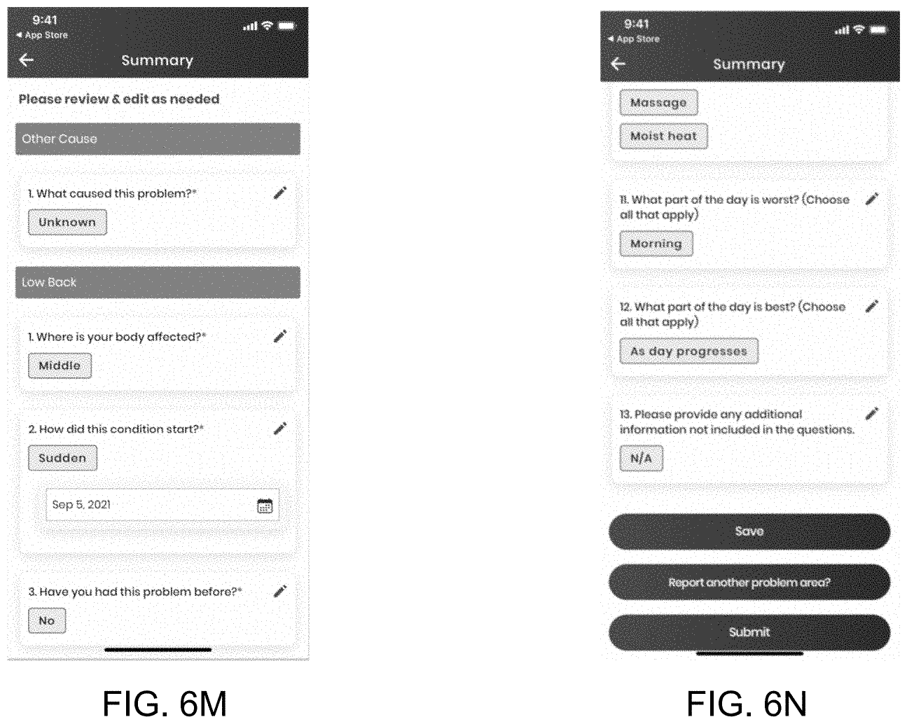

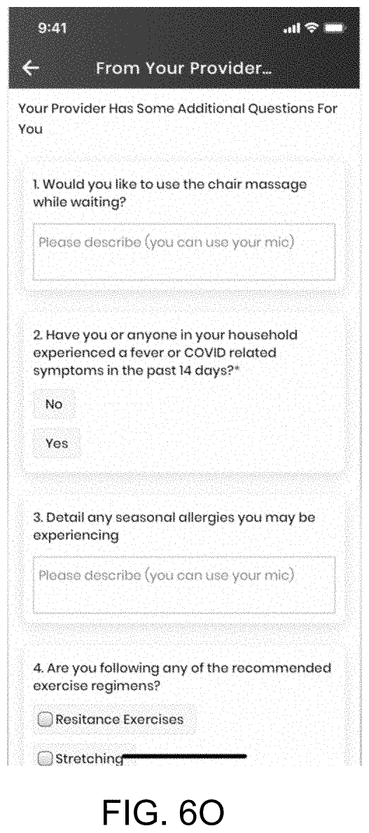

At 212, the mobile device may generate a GUI with the plurality of questions received from the cloud server. For example, the GUI may include one or more of the screenshots shown in FIGS. 6A-6O. The mobile device may display the data that identifies the healthcare facility and may prompt the user to confirm that they are at the correct healthcare facility. The mobile device may also prompt the user to enter responses to the plurality of questions received from the cloud server. Once the user has entered responses to all of the questions, the mobile device may transmit the responses, a unique identifier of the mobile device (e.g., an electronic serial number (ESN), an international mobile equipment identity (IMEI), and/or a mobile equipment identifier (MEID)), and/or a timestamp at which the responses were transmitted to the cloud server at 214, and the procedure 200 may end at 216.

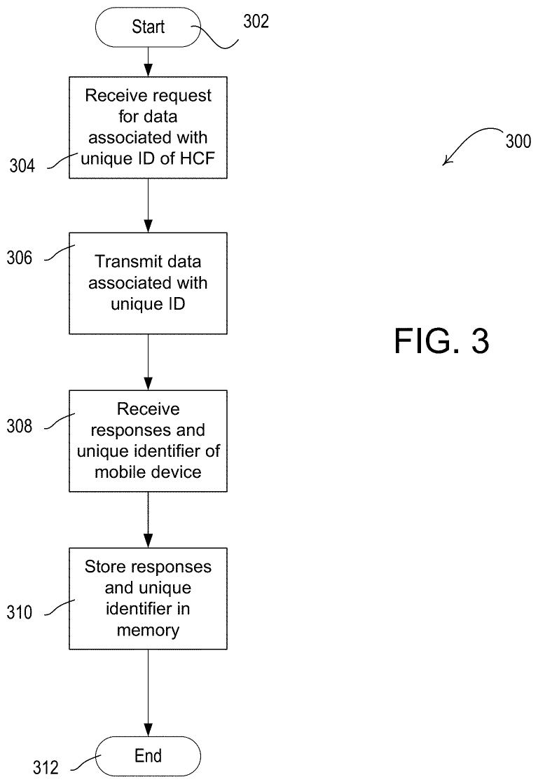

FIG. 3 is a flowchart of an example procedure 300 for checking in a patient to a healthcare facility. The procedure 300 may be performed by a server, such as the cloud server 112 shown in FIG. 1, and/or another device in the load control system. Though the procedure 300 may be described herein as being performed by a single device, such as a server, the procedure 300 may be distributed across multiple devices. In an example, the procedure 300 may be performed by a cloud server and/or a local server.

The procedure 300 may begin at 302. At 304, the server may receive a request for data associated with a unique ID of a healthcare facility. For example, the server may receive the request from a mobile device. The unique ID of the healthcare facility may be determined based on a QR code that may be located within the healthcare facility. After receiving the request from the mobile device, the server may transmit data associated with the unique ID of the healthcare facility to the mobile device at 306. For example, the data associated with the unique ID of the healthcare facility may include data that identifies the healthcare facility and/or a plurality of questions to be answered by a user of the mobile device. The plurality of questions may include one or more facility-specific questions.

At 308, the server may receive responses to the plurality of questions and a unique ID of the mobile device. For example, the responses may include responses to the facility specific questions. The server may also receive a timestamp at which the responses were transmitted by the mobile device. The server may store the responses, the unique ID of the mobile device, and/or the timestamp in memory at 310. The server may also store an indication of the type of the unique ID of the mobile device (e.g., whether the unique ID of the mobile device is an IMEI, an ESN, an MEID, etc.) in memory. Additionally and/or alternatively, the server may transmit the responses, the unique ID of the mobile device, and/or the timestamp to a second server (e.g., a local server), which may then store the information in memory. The procedure 300 may end at 312.

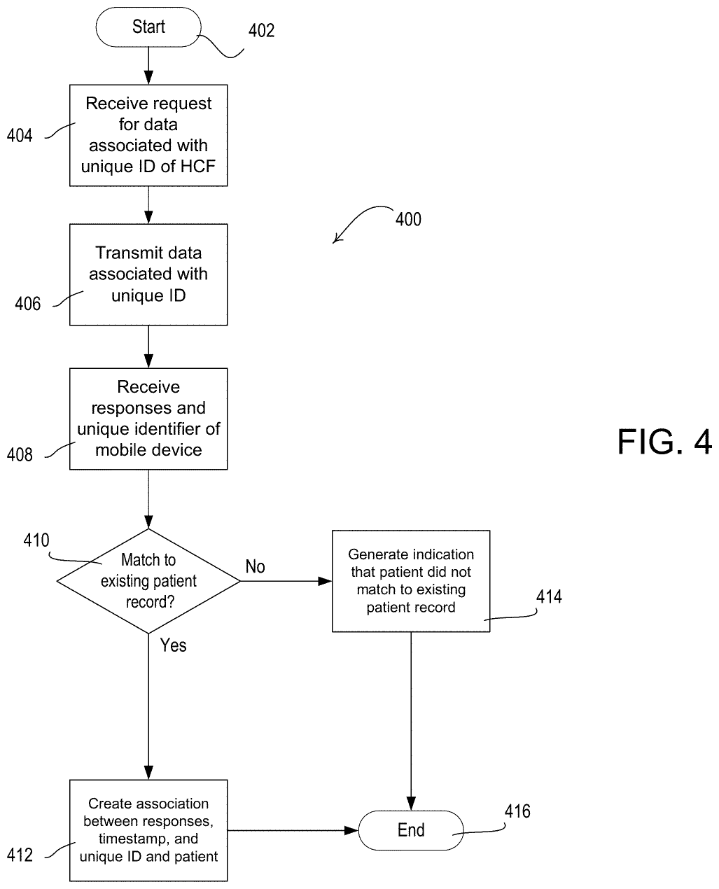

FIG. 4 is a flowchart of an example procedure 400 for checking in a patient to a healthcare facility. The procedure 400 may be performed by a server, such as the cloud server 112 shown in FIG. 1, and/or another device in the load control system. Though the procedure 400 may be described herein as being performed by a single device, such as a cloud server, the procedure 400 may be distributed across multiple devices. In an example, the procedure 400 may be performed by a cloud server and/or a local server.

The procedure 400 may begin at 402. At 404, the server may receive a request for data associated with a unique ID of a healthcare facility. For example, the server may receive the request from a mobile device. The unique ID of the healthcare facility may be determined based on a QR code that may be located within the healthcare facility. After receiving the request from the mobile device, the server may transmit data associated with the unique ID of the healthcare facility to the mobile device at 406. For example, the data associated with the unique ID of the healthcare facility may include data that identifies the healthcare facility and/or a plurality of questions to be answered by a user of the mobile device. The plurality of questions may include one or more facility-specific questions. The cloud server may access the data via a local server.

At 408, the server may receive responses to the plurality of questions and a unique ID of the mobile device. For example, the responses may include responses to the facility specific questions. The server may also receive a timestamp at which the responses were transmitted by the mobile device. The server may transmit the responses, the unique ID of the mobile device, and/or the timestamp to a second server (e.g., a local server), which may then store the information in memory. At 410, the local server may determine whether the user of the mobile device matches to an existing patient record based on the responses to the plurality of questions. For example, the responses may include the user's email address, date of birth (DOB), and/or phone number. The local server may access a database (e.g., which may reside on a memory of the local server) that includes one or more associations between patient records and email addresses, DOBs, and/or phone numbers. For example, a first patient record may be associated with an email address “johnsmith@gmail.com,” a DOB of Jan. 1, 1960, and a phone number 201-555-0123. A second patient record may be associated with an email address “janedoe@gmail.com,” a DOB of Dec. 12, 1975, and a phone number 201-555-9876. The local server may access the database and may determine whether the responses provided by the user match an existing patient record.

The local server may use a hierarchy of information to determine whether the responses match a patient record. For example, the local server may first determine whether the email address provided by the patient matches an email address in an existing patient record. If the local server determines that the email address matches with an email address in an existing patient record, the local server may then determine whether the DOB provided by the patient matches the existing patient record associated with the email address. If the DOB provided by the patient matches the existing patient record, the local server may determine that the user of the mobile device matches with the existing patient record, and the local server may create an association between the responses to the plurality of questions, the unique ID of the mobile device, and/or a timestamp (e.g., the timestamp at which the responses were transmitted by the mobile device and/or a timestamp at which the responses were received by the server) and the patient associated with the existing patient record at 412. Alternatively, a provider or staff member at the healthcare facility may (e.g., manually) create the association between the responses, the unique ID of the mobile device, and the timestamp and the patient associated with the existing patient record. The procedure 400 may end at 416.

If the email address provided by the patient does not match with any patient record, the local server may then determine whether the phone number provided by the patient matches with an existing patient record. If the local server determines that the phone number matches with a phone number in an existing patient record, the local server may then determine whether the DOB provided by the patient matches the existing patient record associated with the phone number. If the DOB provided by the patient matches the existing patient record, the local server may determine that the user of the mobile device matches with the existing patient record, and the local server may create an association between the responses to the plurality of questions, the unique ID of the mobile device, and/or a timestamp (e.g., the timestamp at which the responses were transmitted by the mobile device and/or a timestamp at which the responses were received by the server) and the patient at 412. Alternatively, a provider or staff member at the healthcare facility may (e.g., manually) create the association between the responses, the unique ID of the mobile device, and the timestamp and the patient associated with the existing patient record. The procedure 400 may end at 422.

If neither the email address nor the phone number provided by the user matches to an existing patient record, or if the email address and/or the phone number match to an existing record but the DOB does not, the local server may generate an indication that the patient does not match to an existing patient record at 414. The local server may then store the responses to the plurality of questions, the unique ID of the mobile device, and/or a timestamp (e.g., the timestamp at which the responses were transmitted by the mobile device and/or a timestamp at which the responses were received by the server) in memory. A provider and/or staff member at the healthcare facility may later determine whether the patient is associated with an existing patient record, or if the patient is a new patient. For example, one or more of the plurality of questions may prompt the user of the mobile device to enter whether the patient is a new patient. If the patient is a new patient, the provider or staff member may create a patient record associated with the patient. The provider or staff member at the healthcare facility may then (e.g., manually) create the association between the responses, the unique ID of the mobile device, and the timestamp and the patient associated with the patient record. The procedure 400 may end at 416.

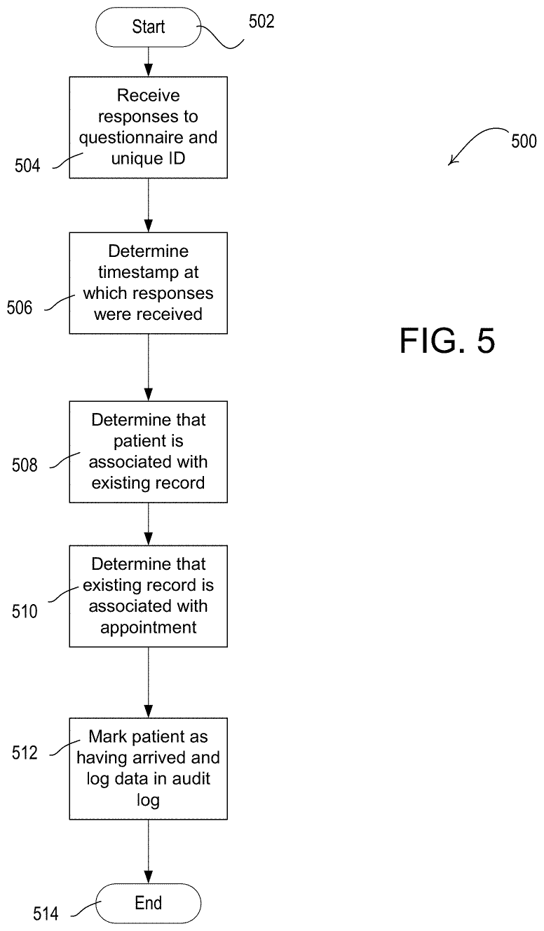

FIG. 5 is a flowchart of an example procedure 500 for checking in a patient to a healthcare facility. The procedure 500 may be performed by a server, such as the local server 114 shown in FIG. 1, and/or another device in the load control system. Though the procedure 500 may be described herein as being performed by a single device, such as a local server, the procedure 500 may be distributed across multiple devices.

The procedure 500 may begin at 502. At 504, the server may receive responses to a plurality of questions (e.g., a questionnaire) and a unique ID of a mobile device. For example, the server may receive the responses and the unique ID from the mobile device (e.g., via a cloud server). The responses may include responses to one or more facility specific questions. At 506, the server may determine a timestamp at which the responses were received. The server may also or alternatively receive a timestamp from the mobile device along with the responses and the unique ID of the mobile device. For example, the timestamp that the server receives from the mobile device may be a timestamp at which the responses were transmitted by the mobile device.

At 508, the server may determine that a patient associated with the responses to the plurality of questions (e.g., a user of the mobile device) is associated with an existing patient record. The server may determine that the patient is associated with an existing patient record based on the responses received from the mobile device. For example, the responses may include an email address, a date of birth (DOB), and/or a phone number associated with the user of the mobile device, and the server may compare the provided email address, DOB, and/or phone number as disclosed at 410 of the procedure 400 shown in FIG. 4.

Referring again to FIG. 5, at 510 the server may determine that the patient record associated with the user of the mobile device is associated with an appointment at the healthcare facility. For example, the patient record may include an indication of a time, date, and/or location of an upcoming appointment for the patient. The server may determine that the timestamp at which the responses were received is within a predetermined amount of time from the upcoming appointment. At 512, the server may mark the patient as having arrived at the healthcare facility and may log data in an audit log. For example, the server may create an indication in the patient record that indicates that the patient has arrived for the appointment. The data that the server logs in the audit log may include, for example, the unique ID of the mobile device and/or a timestamp (e.g., the timestamp at which the responses were received and/or the timestamp at which the responses were transmitted by the mobile device). The audit log may be stored in a memory of the local server, and/or on another device that is accessible by the local server. Once the data has been logged on the audit log, the procedure 500 may end at 514.

FIG. 6A is an example QR code 602 that may be used as part of a procedure for checking in a patient to a healthcare facility. For example, the QR code 602 may be used as part of one or more of the procedures 200, 300, 400, and/or 500 described herein. The QR code 602 may be generated prior to the procedure being performed, and may be displayed within the healthcare facility. For example, the QR code 602 may be the QR code 126 shown in FIG. 1A. A patient may scan the QR code 602 using a mobile device associated with the patient prior to the patient checking in to the healthcare facility.

FIGS. 6B-6O are example graphical user interfaces (GUIs) that may be displayed by a mobile device as part of a procedure for checking in a patient to a healthcare facility. For example, the mobile device may be associated with the patient or with the healthcare facility. The GUIs may be displayed after the patient has scanned a QR code (e.g., the QR code 602 shown in FIG. 6A) that is displayed in the healthcare facility. The GUIs may be displayed using a mobile application running on the mobile device. As disclosed herein, the mobile device may comprise a memory that comprises a computer-readable storage media and/or machine-readable storage media that maintains network information and/or computer-executable instructions for performing as described herein. For example, the memory may comprise computer-executable instructions and/or machine-readable instructions that include one or more portions of the procedures described herein.

In an example, the patient may arrive at the healthcare facility, and may open an app on a mobile device associated with the patient. If the patient has not previously used the app, the app may prompt the patient to create an account with an email address and password (FIG. 6B) and enter identifying information about the patient (FIG. 6C), which may include the patient's name, DOB, phone number, email address, and/or ZIP code. Alternatively, if the patient has previously used the app, the app may prompt the patient to sign in using their email address and password.

Once the patient has signed into the app, the app may display a home page (FIG. 6D), where the patient may choose from one or more selections. For example, as shown in FIG. 6D, the app may prompt the patient to view their patient profile or their visit history, or to check in for an appointment. If the patient selects the check in option, the app may display information about the healthcare facility (FIG. 6E), for example including the address of the healthcare facility, the name of the healthcare facility, and/or the current date. As shown in FIG. 6E, the app may prompt the patient to confirm that the information about the healthcare facility is correct.

If the patient confirms that the information displayed is correct, the app may prompt the user to scan a QR code (e.g., the QR code 602) using a camera of the mobile device (FIG. 6F). For example, the QR code may be physically displayed in the healthcare facility. After the patient scans the QR code, the app may determine a unique ID for the healthcare facility based on the QR code, and may transmit the unique ID for the healthcare facility and a request for data associated with the unique ID for the healthcare facility to a cloud server. The cloud server may receive the request for data associated with the unique ID for the healthcare facility, and may transmit the data associated with the unique ID to the mobile device. For example, the data may include a plurality of questions (e.g., that may include one or more facility-specific questions) and/or data that identifies the healthcare facility.

The mobile device may receive the data from the cloud server. The app may display the data that identifies the healthcare facility, and may prompt the patient to confirm that they are at the correct location (FIG. 6G). If the patient confirms that they are at the correct location, the app may prompt the user to select the type of problem that caused their visit to the healthcare facility (FIG. 6H). The app may then prompt the patient to select a visit type (FIG. 6I). For example, the app may prompt the patient to select whether the visit is a new visit or a follow-up visit.

Once the patient has selected the visit type, the app may prompt the patient to provide responses to the plurality of questions received from the cloud server (FIGS. 6J-6L). For example, the app may prompt the patient to select whether the cause of the problem is known or unknown (FIG. 6J) and/or the part(s) of the body in which the problem is located (FIG. 6K), and/or to answer one or more questions regarding the problem (FIG. 6L). Once the patient has entered their responses, the app may prompt the user to review and submit their responses (FIGS. 6M and 6N). The app may also prompt the user to answer one or more facility-specific questions (FIG. 6O). Once the patient has submitted responses to the questions, the mobile device may transmit the responses, a unique ID of the mobile device, and/or a timestamp at which the responses were transmitted to the cloud server.

Although features, elements, and functions are described above in particular combinations, a feature, element, or function is used alone or in any combination with the other features, elements, or functions. Various presently unforeseen or unanticipated alternatives, modifications, variations, or improvements may be subsequently made that are also intended to be encompassed by the following claims.

The methods described herein are implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), removable disks, and optical media such as CD-ROM disks, and digital versatile disks (DVDs).

What is claimed is:

1. A system for registering a patient upon arrival at a healthcare facility, the system comprising:
a mobile device comprising:
a communication circuit; and
a processor configured to:
receive a scan of a QR code associated with the healthcare facility;
determine a unique identifier of the healthcare facility based on the QR code;
transmit, via the communication circuit, the unique identifier of the healthcare facility and a request for data associated with the QR code to a cloud server;
receive, via the communication circuit, the data associated with the QR code and a plurality of questions;
generate a graphical user interface (GUI) via a display of the mobile device that comprises the plurality of questions; and
transmit, via the communication circuit, responses to the plurality of questions and a unique identifier associated with the mobile device to the cloud server; and
a local server comprising:
a communication circuit; and
a processor configured to:
receive, via the communication circuit, the responses to the plurality of questions and the unique identifier of the mobile device from the cloud server; and
store the responses and the unique identifier of the mobile device in memory.

2. The system of claim 1, wherein the processor of the local server is further configured to store a timestamp indicating a time at which the responses to the plurality of questions and the unique identifier of the mobile device were received from the cloud server.

3. The system of claim 1, wherein the unique identifier of the mobile device comprises one or more of an electronic serial number (ESN), an international mobile equipment identity (IMEI), or a mobile equipment identifier (MEID).

4. The system of claim 1, further comprising the cloud server, and wherein after the local server has received the responses to the plurality of questions and the unique identifier of the mobile device, the cloud server is configured to delete the responses to the plurality of questions and the unique identifier of the mobile device from memory.

5. The system of claim 1, wherein the plurality of questions comprise one or more facility-specific questions, and wherein the responses comprise responses to the facility-specific questions.

6. The system of claim 1, wherein the processor of the local server is further configured to:
determine that the responses match to information associated with an existing patient record; and
create an association between the responses and the existing patient record.

7. The system of claim 6, wherein the responses comprise one or more of an email address associated with a user of the mobile device, a date of birth (DOB) of the user, and a phone number associated with the user, and wherein the processor of the local server is configured to determine that the responses match to the information associated with the existing patient record based on one or more of the email address, the DOB, and the phone number.

8. The system of claim 1, wherein the processor of the local server is further configured to:
determine that the responses do not match to information associated with an existing patient record;
generate an indication that the responses do not match to information associated with an existing patient record.

9. A method for registering a patient upon arrival at a healthcare facility, the method comprising:
receiving a request for data associated with a unique identifier of the healthcare facility;
transmitting the data associated with the unique identifier of the healthcare facility to a mobile device, wherein the data comprises a plurality of questions;
receiving responses to the plurality of questions and a unique identifier associated with the mobile device; and
storing the responses and the unique identifier of the mobile device in memory.

10. The method of claim 9, wherein the plurality of questions comprise one or more facility-specific questions, and wherein the responses comprise responses to the one or more facility-specific questions.

11. The method of claim 9, wherein the data further comprises information that identifies the healthcare facility.

12. The method of claim 9, further comprising storing a timestamp at which the responses to the plurality of questions were received in memory.

13. The method of claim 9, further comprising:
determining that the responses match to information associated with an existing patient record; and
creating an association between the responses and the existing patient record.

14. The method of claim 13, wherein the responses comprise one or more of an email address associated with a patient, a date of birth (DOB) of the patient, and a phone number associated with the patient, and wherein the processor of the local server is configured to determine that the responses match to the information associated with the existing patient record based on one or more of the email address, the DOB, and the phone number.

15. The method of claim 9, further comprising:
determining that the responses do not match to information associated with an existing patient record; and
generating an indication that the responses do not match to information associated with an existing patient record.

16. A method for registering a patient upon arrival at a healthcare facility, the method comprising:
receiving responses to a plurality of questions and a unique identifier associated with a mobile device;
determining a timestamp at which the responses and the unique identifier were received;
determining that the responses are associated with an existing patient record;
determining that the existing patient record is associated with an appointment that is scheduled within a predetermined amount of time of the timestamp; and
logging the responses to the plurality of questions, the unique identifier, and the timestamp in an audit log.

17. The method of claim 16, further comprising marking the patient as having arrived at the healthcare facility.

18. The method of claim 16, wherein the responses comprise one or more of an email address associated with the patient, a date of birth (DOB) of the patient, and a phone number associated with the patient, and wherein determining that the responses are associated with an existing patient record comprises determining that one or more of the email address associated with the patient, the DOB of the patient, and the phone number associated with the patient match with a stored date of birth, a stored email address, or a stored phone number in the patient record.

19. A system for registering a patient upon arrival at a healthcare facility, the system comprising:
a mobile device comprising:
a communication circuit; and
a processor configured to:
receive a scan of a QR code associated with the healthcare facility;
determine a unique identifier of the healthcare facility based on the QR code;
transmit, via the communication circuit, the unique identifier of the healthcare facility and request for data associated with the QR code to a cloud server;
receive, via the communication circuit, the data associated with the QR code and a plurality of questions;
generate a graphical user interface (GUI) via a display of the mobile device that comprises the plurality of questions; and
transmit responses to the plurality of questions and a unique identifier associated with the mobile device to the cloud server via the communication circuit; and
a local server comprising:
a communication circuit; and
a processor configured to:
receive, via the communication circuit, the responses to the plurality of questions and the unique identifier of the mobile device from the cloud server; and
determine a timestamp at which the responses and the unique identifier of the mobile device were received;
determine that the responses match to information associated with an existing patient record;
determine that the existing patient record is associated with an appointment that is scheduled within a predetermined amount of time of the timestamp; and
log the responses to the patient questionnaire, the unique identifier, and the timestamp in an audit log.
